SEQUENCE LISTING (6 sequences)

SEQ ID NO: 1 - RNA, 87 nt, Influenza A virus
agcaaaagcagggugacaaagacauaauggauccaaacacugugucaagcuuucagguagauugcuuucuuuggcauguccgcaaac

SEQ ID NO: 2 - DNA, 122 bp, Artificial Sequence
  synthesized DNA
taatacgactcactataggccatcctgtttttttcccttttttttttctttttttttttttttttttttttttttttttttttctcctttttttttcctctttttttccttttctttccttt

SEQ ID NO: 3 - DNA, 16 bp, Artificial Sequence
  synthesized DNA
taatacgactcactat

SEQ ID NO: 4 - DNA, 10 bp, Artificial Sequence
  synthesized DNA
aggccatcct

SEQ ID NO: 5 - DNA, 96 bp, Artificial Sequence
  synthesized DNA
gtttttttcccttttttttttctttttttttttttttttttttttttttttttttttctcctttttttttcctctttttttccttttctttccttt

SEQ ID NO: 6 - RNA, 115 nt, Hepatitis C virus
uaauacgacucacuauagguggcuccaucuuagcccuagucacggcuagcugugaaagguccgugagccgcuugacugcagagagugcugauacuggccucucugcagaucaagu

(19) United States
(12) Patent Application Publication
(10) Pub. No.: US 2022/0396797 A1
(43) Pub. Date: Dec. 15, 2022

(54) RIG-I AGONIST AND ADJUVANT FORMULATION FOR TUMOR TREATMENT

(71) Applicant: INFECTIOUS DISEASE RESEARCH INSTITUTE, Seattle, WA (US)

(72) Inventors: Malcolm S. DUTHIE, Sammamish, WA (US); Steven G. REED, Bellevue, WA (US)

(21) Appl. No.: 17/777,255

(22) PCT Filed: Nov. 13, 2020
(86) PCT No.: PCT/US2020/060583

§ 371 Date: May 16, 2022

Related U.S. Application Data
(60) Provisional application No. 62/936,228, filed on Nov. 15, 2019.

(51) Int. Cl. C12N 15/117; A61P 35/00; A61K 9/107

(52) U.S. Cl. CPC C12N 15/117; A61P 35/00; A61K 9/1075; C12N 2310/17; C12N 2310/531

(57) ABSTRACT

Cancer immunotherapies effective for treating tumors are disclosed. A cytosolic receptor that detects viral ribonucleic acid (RNA), retinoic-acid-inducible protein 1 (RIG-I), is activated by a synthetic double-stranded oligonucleotide pUUC Auk. In one implementation, RIG-I is activated by a single-stranded oligonucleotide agonist formulated with a squalene emulsion (SE) adjuvant. Activation of RIG-I with pUUC Auk results in slower tumor growth. Formulation with a SE adjuvant increases the ability of RIG-I agonists to slow tumor growth. The novel agonist and formulations provided herein are effective for slowing the growth of new tumors and reducing further growth of established tumors. RIG-I agonist/adjuvant formulations may be administered to a subject by any of multiple routes including intratumoral injection.

### BACKGROUND

The intrinsic immune system provides defenses against viral infections through non-self recognition mechanisms that initiate multiple defensive immune responses. These responses include pro-inflammatory cytokine production, recruitment of other immune cells (e.g., macrophages and natural killer cells), and immunogenic cell death (pyroptosis). Intrinsic immune responses can be initiated in virally infected cells by pattern-recognition receptors such as the intracellular protein retinoic-acid-inducible protein 1 (RIG-I). RIG-I detects certain patterns in double-stranded ribonucleic acid (dsRNA) sequences. Many of these patterns are found in the RNA of viruses. Once activated by detection of a dsRNA sequence, RIG-I changes confirmation which initiates a cascade of immune responses that can ultimately lead to cell death and inflammatory responses.

Cancer immunotherapy uses these defensive functions of the intrinsic immune system against cancer. The immune responses evolved for combating viral infections are intentionally triggered in tumors or cancerous cells by delivery of synthetic, non-infectious RNA sequences that activate RIG-I receptors. (Elion, D. L., & Cook, R. S. (2018). Harnessing RIG-I and intrinsic immunity in the tumor microenvironment for therapeutic cancer treatment. Oncotarget, 9(48), 29007-29017.) The goal of this therapy is to activate RIG-I so that it will cause the intrinsic immune system to attack cancerous cells as if they were virally infected cells. While this aspect of the immune system is powerful it is also poorly understood. It is not currently possible to accurately predict which RNA sequences will activate RIG-I or how to best formulate such sequences to induce an immune response against tumors.

It is with respect to these and other considerations that the following disclosure is made.

### SUMMARY

This disclosure provides a novel RIG-I agonist and identifies an unexpected adjuvant formulation. Both the novel agonist and the adjuvant formulation exhibit the ability to slow tumor growth. Agonists are chemicals that bind to and activate a receptor molecule such as RIG-I. Adjuvants are pharmacological or immunologic agents that modify the effect of other agents. Adjuvants are used to create stronger immune response to an agonist.

The agonists for RIG-I are oligonucleotide sequences that have structures similar to viral RNA such as an uncapped 5′ di/triphosphate end and a short blunt-ended double-stranded portion. However, not every oligonucleotide with these characteristics can activate RIG-I. The novel agonist provided in this disclosure is pUUC Auk which is a single-stranded deoxyribonucleic acid (DNA) molecule with 5′ triphosphate, a single hairpin region, and poly-T regions. Treatment with pUUC Auk is shown to slow tumor growth. The ability of pUUC Auk to slow tumor growth is dose dependent; larger doses result in a greater slowing of tumor growth.

The adjuvant identified in this disclosure as increasing the immune response to RIG-I agonists is a squalene emulsion (SE). Squalene, a natural organic compound with the chemical formula C₃₀H₅₀, by itself is not an adjuvant, but emulsions of squalene with surfactants are known to enhance immune response. The mechanism by which SE enhances immune response is poorly understood. Many vaccines that benefit from formulation with SE include agonists that activate cell-surface receptors. However, RIG-I is located inside cells and cells are not believed to have any mechanism for uptake of SE. Accordingly, it is unexpected that a SE adjuvant increases the immunogenicity of an agonist targeting a cytosolic receptor. Addition of SE as an adjuvant to formulations that include a RIG-I agonist increase the immune response initiated by RIG-I. This allows lower doses of agonists to effectively slow tumor growth.

The formulations provided in this disclosure contain a RIG-I agonist which may be pUUC Auk or another agonist and optionally comprise an adjuvant. The adjuvant may be SE or another adjuvant such as a nanostructured lipid carrier (NLC), a glucopyranosyl lipid adjuvant in an aqueous formulation (GLA-AF), an aluminum adjuvant (alum), or another adjuvant. This disclosure also provides methods of administering any of the above formulations to a subject to stimulate an immune response against cancerous cells such as a tumor. The cancer cells may be cells from any type of cancer such as liver, head, neck, pancreatic, melanoma, etc. Suitable routes of administration for the formulations in this disclosure include, but are not limited to, intratumoral, intravenous, subcutaneous, intradermal, intraperitoneal, intracranial, and intrathecal.

This Summary is provided to introduce a selection of concepts in a simplified form that are further described below in the Detailed Description. This Summary is not intended to identify key features or essential features of the claimed subject matter nor is it intended to be used to limit the scope of the claimed subject matter.

### BRIEF DESCRIPTION OF THE DRAWINGS

The Detailed Description is set forth with reference to the accompanying figures. In the figures, the left-most digit(s) of a reference number identifies the figure in which the reference number first appears. Although the following figures depict various aspects of the invention, the invention is not limited to any depiction in the figures.

FIG. 1 shows a predicted structure for a single-stranded RNA molecule 3p-hpRNA that includes multiple double-stranded regions. 3p-hpRNA is a RIG-I agonist.

FIG. 2 shows a predicted structure for a single-stranded DNA molecule pUUC Auk with a single hairpin region, a first linear region to the 5′-side of the hairpin region, and a second linear region to the 3′-side of the hairpin region. pUUC Auk is a RIG-I agonist.

FIG. 3 shows a predicted structure for a single-stranded RNA molecule XRNA that includes multiple double-stranded regions. XRNA is used as an RNA control because it does not activate RIG-I.

FIG. 4 is a graph showing tumor growth in mice inoculated with B16 melanoma cells. Tumors grow more slowly in mice treated with the RIG-I agonist 3p-hpRNA. This demonstrates that RIG-I activation can slow tumor growth.

FIG. 5 is a graph showing tumor growth in mice inoculated with B16 melanoma cells. Tumors grew more slowly in mice treated with the RIG-I agonist pUUC Auk. The agonist pUUC Auk slows tumor growth in a dose-dependent manner. This demonstrates that treatment with the agonist pUUC Auk can slow tumor growth.

FIG. 6 is a graph showing tumor growth in mice inoculated with B16 melanoma cells. Treatment with pUUC Auk slowed tumor growth in a dose-dependent manner. The effects of the agonist pUUC AuK were increased when formulated with a squalene emulsion (SE) adjuvant. This demonstrates that formulation with SE increases the efficacy of pUUC Auk.

FIG. 7 is a graph showing the growth of established tumors in mice inoculated with B16 melanoma cells. Intratumoral injection with pUUC Auk formulated with SE slowed tumor growth. This demonstrates that a formulation comprising pUUC Auk and SE is effective at limiting further growth of established tumors.

### DETAILED DESCRIPTION

The receptor targeted by agonists and agonist/adjuvant formulations of this disclosure is the RNA helicase RIG-I which is a cytoplasmic sensor expressed in the majority of cell types. RIG-I is part of the RIG-I-like receptor (RLR) family, which also includes MDA5 and LGP2, and functions as a pattern recognition receptor that is a sensor for viral RNA. RIG-I is encoded in humans by the DDX58 gene. Upon activation, RIG-I recruits the adaptor mitochondrial antiviral signaling protein (MAVS, or IPS-1/VISA/Cardif) which then triggers signaling cascades that lead to the production of type I interferons (IFNs) and pro-inflammatory cytokines. RIG-I signaling directly promotes killing of cells in which RIG-I has been activated through three distinct modes of action: intrinsic apoptosis, extrinsic apoptosis, and pyroptosis.

RIG-I agonist therapeutics show promise for treating cancer, however, RIG-I-based therapeutic strategies face multiple challenges, such as designing highly specific and stable agonists, and developing efficient agonist delivery modes while avoiding uncontrolled release of pro-inflammatory cytokines. Additionally, difficulty identifying in advance which oligonucleotide sequences will be recognized by RIG-I combined with the unpredictable interaction between agonist and adjuvant makes it challenging to identify specific molecules and formulations that are effective cancer therapies.

### RIG-I Agonists

RIG-I agonists are molecules that bind to RIG-I and induce a conformational change. Agonists RIG-I evolved to recognize are typically short (<4000 nucleotides (nt)) 5′ triphosphate uncapped double-stranded (ds) or single-stranded (ss) RNA sequences obtained from the genomes of infecting viruses. RIG-I detects intracellular RNA viruses by the presence of uncapped dsRNA modified with a 5′-triphosphate (5′-3pRNA) or 5′-diphosphate motif (5′-2pRNA). RIG-I can be induced by 5′-3pRNA as short as 19 nt. It is believed that a double-stranded structure is necessary for activation. However, there is still uncertainty and conflicting data regarding which types of RNA structures will activate RIG-I. (Baum, A., & García-Sastre, A. (2010) Induction of type I interferon by RNA viruses: cellular receptors and their substrates. Amino acids, 38(5), 1283-1299.) Without being bound by theory, it is believed that a poly-uridine (U) rich motif in the RNA ligand is involved in activating RIG-I. RNA strands with poly-U motifs as short as 13 nt have activated RIG-I. (Kell, A., et al. (2015) Pathogen-Associated Molecular Pattern Recognition of Hepatitis C Virus Transmitted/Founder Variants by RIG-I Is Dependent on U-Core Length. Journal of virology, 89(21), 11056-11068.)

The RIG-I agonist may be a natural or synthetic double- or single-stranded oligonucleotide with a 5′ phosphate group. For example, the RIG-I agonist may be an uncapped single-stranded RNA molecule containing a stable duplex structure and terminated with a 5′-triphosphate or diphosphate group. Some RIG-I agonists can form a stem loop structure. In some implementations, the RIG-I agonist is itself recognized by the RIG-I receptor itself or can produce a recognizable sequence by in vivo modification. For example, the RIG-I agonist may be a replication intermediate

“Oligonucleotide,” as used herein, refers to short, generally single-stranded synthetic polynucleotides that are generally, but not necessarily, less than about 200 nucleotides in length. A “polynucleotide,” “nucleotide,” or “nucleic acid,” as used interchangeably herein, refer to polymers of nucleotides of any length, including DNA, RNA, and hybrid DNA-RNA molecules. The nucleotides can be, for example, deoxyribonucleotides, ribonucleotides, modified nucleotides or bases, and/or their analogs, or any substrate that can be incorporated into a polymer by DNA or RNA polymerase, or by a synthetic reaction. A polynucleotide may comprise modified nucleotides, such as methylated nucleotides and their analogs. If present, modification to the nucleotide structure may be imparted before or after assembly of the polymer.

As used herein, a “RIG-I agonist” refers to any natural or synthetic molecule that directly or indirectly interacts with the RIG-I receptor causing conformational changes in the receptor that induces an immune response. The immune response may be induced by the RIG-I agonist alone or in the presence of one or more adjuvants. Thus, a RIG-I agonist may be any molecule that interacts with the regular receptor to trigger a signaling cascade which leads to the production of type I IFNs and pro-inflammatory cytokines. Specific RIG-I agonists discussed in this disclosure are 3p-hpRNA and pUUC AuK. RIG-I agonist also encompasses any variations, modifications, or similar molecules to 3p-hpRNA and pUUC AuK that retain the ability to activate RIG-I.

Suitable synthetic methods can be used alone, or in combination with one or more other methods (e.g., recombinant DNA or RNA technology), to produce a synthetic oligonucleotide that functions as a RIG-I agonist. Suitable methods for de novo synthesis are well-known in the art and can be adapted for particular applications. Such methods include chemical synthesis using suitable protecting groups such as CEM, the β-cyanoethyl phosphoramidite method, and the nucleoside H-phosphonate method. These chemistries can be performed or adapted for use with automated nucleic acid synthesizers that are commercially available.

Additional suitable synthetic methods are disclosed in Uhlmann et al. (1990) Chem Rev 90:544-84, and Goodchild J (1990) Bioconjugate Chem 1:165. Nucleic acid synthesis can also be performed using suitable recombinant methods that are well-known and conventional in the art, including cloning, processing, and/or expression of polynucleotides and gene products encoded by such polynucleotides. DNA shuffling by random fragmentation and PCR reassembly of gene fragments and synthetic polynucleotides are examples of known techniques that can be used to design and engineer polynucleotide sequences. Site-directed mutagenesis can be used to alter nucleic acids and the encoded proteins, for example, to insert new restriction sites, alter glycosylation patterns, change codon preference, produce splice variants, introduce mutations and the like. Suitable methods for transcription, translation, and expression of nucleic acid sequences are known and conventional in the art.

The presence and/or quantity of one or more modified nucleotides in an oligonucleotide can be determined using any suitable method. For example, a RIG-I agonist can be digested to monophosphates (e.g., using nuclease PI) and dephosphorylated (e.g., using a suitable phosphatase such as calf intestinal alkaline phosphatase (CIAP)), and the resulting nucleosides analyzed by reversed-phase high-performance liquid chromatograph (HPLC). Optionally, RNA molecules may include one or more modified nucleotides

If desired, the synthetic RIG-I agonist oligonucleotides can be screened or analyzed to confirm their therapeutic and prophylactic properties using various in vitro or in vivo testing methods that are known to those of skill in the art. For example, formulations comprising RIG-I agonist can be tested for their effect on activation of the RIG-I receptor. This testing may determine if a potential agonist is capable of in vivo or in vitro activation of the RIG-I receptor to trigger the signaling cascade that lead to the production of type I IFNs and pro-inflammatory cytokines.

### Adjuvants

A formulation of a RIG-I agonist may also include an adjuvant. In some implementations, the adjuvant and the RIG-I agonist are co-formulated in a single vial for use as a therapeutic agent. In some implementations, the adjuvant acts separately from the RIG-I agonist after administration. For example, but without being restricted to a specific theory, the adjuvant acts on the contents of a cell that is damaged by the action of the RIG-I agonist. In some implementations, more than one adjuvant is included in the formulation. In some implementations, the adjuvant is a nanostructured lipid carrier (NLC), a squalene emulsion (SE), a GLA-AF (aqueous formulation), or an aluminum adjuvant (alum).

In some implementations, the formulation is an emulsion. In some implementations, the emulsion is the adjuvant. In some implementations, a formulation of the RIG-I agonist and the adjuvant is chosen that allows the formulation to be frozen and/or lyophilized in a single vial.

In some implementations, the adjuvant is an immunostimulatory adjuvant. Immunostimulatory adjuvants can be adjuvants that directly act on the immune system such as, for example, a cytokine, a TLR ligand or a microbial toxin. Adjuvants for use in compositions that modify the immune response are well known in the art. Thus, adjuvants for use in compositions described herein may comprise one or more of an immunostimulatory adjuvant, a delivery adjuvant, an inorganic adjuvant, or an organic adjuvant. Non-limiting examples of adjuvants for use in compositions described herein can be found, inter alia, in Barouch D. H., 2008, Nature, 455(7213):613-9; Morrow et al., 2008, AIDS, 22(3):333-8; and McGeary et al., 2003, Peptide Sci., 9(7):405-181.

In some implementations, the adjuvant is an oil-in-water emulsion. An immiscible oil and water mixture can be emulsified using an appropriate surfactant to create an oil-in-water (o/w) emulsion (oil droplets surrounded by an aqueous bulk phase). Some emulsions are self-emulsifying, while others require various levels of energy input via temperature increase, blending, sonication, high-pressure homogenization, or other methods. Oil in water emulsions are stable for years at ambient temperature and can be frozen. The RIG-I agonist is added after emulsification for stability of the RNA and to facilitate manufacture.

The oil may be natural or synthetic and may be mineral or organic. Examples of mineral and organic oils will be readily apparent to the skilled artisan. In some implementations, the formulation is an emulsion of oil-in-water wherein the RIG-I agonist nucleic acid is incorporated in the oil phase. In some implementations, in order for an oil-in-water composition to be suitable for human administration, the oil phase of the emulsion system comprises a metabolizable oil. The meaning of the term metabolizable oil is well known in the art. Metabolizable can be defined as “being capable of being transformed by metabolism” (Dorland's Illustrated Medical Dictionary, W. B. Saunders Company, 25th edition (1974)).

The oil may be any plant oil, vegetable oil, fish oil, animal oil or synthetic oil, which is not toxic to the recipient and is capable of being transformed by metabolism. Nuts (such as peanut oil), seeds, and grains are common sources of vegetable oils. Synthetic oils may also be used such as synthetic squalene. A description of synthetic squalene as vaccine adjuvant can be found in Adlington et al., 2016, Biomacromolecules, 17:165-172.) Illustrative metabolizable oils include, but are not limited to, squalene, soybean oil, sesame oil and caprylic/capric acid triglycerides (e.g., MIGLYCOL 810 oil). In one implementation, the metabolizable oil comprises squalene or synthetic squalene. In another implementation, the metabolizable oil comprises one or more yeast-derived isoprenoids, such as yeast-derived squalene or related isoprenoid structure derived from yeast.

In some implementations, the adjuvant is a squalene emulsion (SE). Squalene (2,6,10,15,19,23-Hexamethyl-2,6,10,14,18,22-tetracosahexaene) is an unsaturated oil which is found in large quantities in shark-liver oil, and in lower quantities in olive oil, wheat germ nil, rice bran oil, and yeast. Squalene is used in vaccine and drug delivery emulsions due to its stability-enhancing effects and biocompatibility. Emulsions containing squalene facilitate solubilization, modified release and cell uptake of adjuvants. However, the squalene used herein can be natural or synthetic. As used herein, SE includes emulsions of natural or synthetic squalene. Squalene is a metabolizable oil by virtue of the fact that it is an intermediate in the biosynthesis of cholesterol (Merck index, 10th Edition, entry no. 8619). Squalene emulsions are efficient adjuvants, eliciting both humoral and cellular immune responses.

In some implementations, the squalene emulsion comprises squalene and a surfactant (also known as an emulsifier or emulsifying agent). There are a number of surfactants specifically designed for and commonly used in biological applications. Such surfactants are divided into four basic types: anionic, cationic, zwitterionic and nonionic. One group of surfactants are the hydrophilic non-ionic surfactants and, in particular, polyoxyethylene sorbitan monoesters and polyoxyethylene sorbitan triesters. These materials are referred to as polysorbates and are commercially available under the mark TWEEN® and are useful for preparing the NLCs. TWEEN® surfactants generally have a hydrophilic-lipophilic balance (HLB) value falling between 9.6 to 16.7. TWEEN® surfactants are commercially available.

Other non-ionic surfactants which can be used are, for example, polyoxyethylene fatty acid ethers derived from lauryl, acetyl, stearyl and oleyl alcohols, polyoxyethylene fatty acids made by the reaction of ethylene oxide with a long-chain fatty acid, polyoxyethylene, polyol fatty acid esters, polyoxyethylene ether, polyoxypropylene fatty ethers, bee's wax derivatives containing polyoxyethylene, polyoxyethylene lanolin derivative, polyoxyethylene fatty glycerides, glycerol fatty acid esters or other polyoxyethylene fatty acid, alcohol or ether derivatives of long-chain fatty acids of 12-22 carbon atoms. In some implementations, the surfactant is Tween. In some implementations, the surfactant is polysorbate 80 (Tween® 80). In some implementations, the emulsifier is lecithin. In some implementations, the squalene emulsion comprises both Tween and lecithin.

In some implementations, the squalene emulsion comprises from about 0.5% v/v squalene to about 10% v/v squalene, including, but not limited to, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3/3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8% and 9.9% v/v. In some implementations, the squalene emulsion comprises Tween in an amount between about 0.25% v/v and about 1% v/v, including but not limited to, 0.3%, 0.35%, 0.4%, 0.45%, 0.5%, 0.55%, 0.6%, 0.65%, 0.7%, 0.75%, 0.8%, 0.85%, 0.9%, 0.95%, and 0.99% v/v. In some implementations, the squalene emulsion comprises lecithin in an amount between about 0.2% v/v and about 3% v/v, including but not limited to, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, and 3.0% v/v. In some implementations, the squalene emulsion comprises about 1% v/v squalene, and/or about 0.2% v/v polysorbate 80 and/or about 1.62 mg/ml lecithin.

In some implementations, the adjuvant is a Nanostructured Lipid Carrier (NLC). Nanostructured lipid carriers can be formulated and then the RIG-I agonist added. In some implementations, the RIG-I agonist RNA when admixed with the NLC is presented on the outside of the NLC. Any NLC known in the art can be used without exception. It will be understood by the skilled practitioner that a NLC is made up of NLC particles. NLCs are described in Beloqui et al., Nanomedicine. NBM 2016; 12: 143-161. NLC particles may comprise (a) an oil core comprising a liquid phase lipid and a solid phase lipid, (b) a cationic lipid, (c) a hydrophobic surfactant (including but not limited to, a sorbitan ester (e.g., sorbitan monoester, diester or triester), and (d) a hydrophilic surfactant. Examples of NLC's are provided in PCT application WO 2018/232257 (PCT/US2018/037783 A1). Compositions are stable and are capable of the delivery of RIG-I formulations, for example, for the generation of an immune response and/or for treatment of cancers/tumors in a subject. In some implementations, the NLC is made up of at least an oil core comprising a mixture of a liquid phase lipid and a solid phase lipid, a cationic component such as a cationic lipid, a hydrophobic surfactant such as a sorbitan ester, and a surfactant (e.g., a hydrophilic surfactant).

In some implementations, the liquid phase lipid (also called “liquid oil” or “liquid phase oil”) is metabolizable, such as a vegetable oil, animal oil, fish oil, or synthetically prepared oil (e.g., squalene). In some implementations, the hydrophobic surfactant is a sorbitan ester (e.g., Span 85, Span 80, or Span 60). In some implementations, the hydrophilic surfactant is a polyethylene glycol, or a polyoxyethylene sorbitan ester (e.g., Tween® 80). In some implementations, the cationic component is a cationic lipid selected from: 1,2-dioleoyloxy-3-(trimethylammonio)propane (DOTAP), 3β-[N—(N′,N′-Dimethylaminoethane)-carbamoyl] Cholesterol (DC Cholesterol), dimethyldioctadecylammonium (DDA), 1,2-Dimyristoyl-3-TrimethylAmmoniumPropane (DMTAP), dipalmitoyl(C16:0)trimethyl ammonium propane (DPTAP), di stearoyltrimethylammonium propane (DSTAP), N-[1-(2,3-dioleyloxy)propyl]-N,N,N-trimethylammonium chloride (DOTMA), N,N-dioleoyl-N,N-dimethylammonium chloride (DODAC), 1,2-dioleoyl-sn-glycero-3-ethylphosphocholine (DOEPC), 1,2-dioleoyl-3-dimethylammonium-propane (DODAP), and 1,2-dilinoleyloxy-3-dimethylaminopropane (DLinDMA), and combinations thereof. In some implementations, a solid oil is included (e.g., Dynasan 114).

In some implementations, the adjuvant for use in the formulation as described herein may be aluminum adjuvants, which are generally referred to as “alum.” Alum adjuvants are based on the following: aluminum oxy-hydroxide; aluminum hydroxyphosphate; or various proprietary salts. Alum adjuvants are advantageous because they have a good safety record, augment antibody responses, stabilize antigens, and are relatively simple for large-scale production. (Edelman 2002 Mol. Biotechnol. 21:129-148; Edelman, R. 1980 Rev. Infect. Dis. 2:370-383.). Alum adjuvants can be used in combination with any of the emulsions, adjuvants or excipients described herein in the formulations.

In some implementations, an immunostimulatory adjuvant such as the Toll-like receptor (TLR) ligand (e.g., a TLR agonist) is used in the formulations. One or more TLR ligands can be suitable as an adjuvant alone or in a combination with one or more additional adjuvant in a composition described herein. TLRs include cell surface transmembrane receptors of the innate immune system that confer early-phase recognition capability to host cells for a variety of conserved microbial molecular structures such as may be present in or on a large number of infectious pathogens. (e.g., Armant et al., 2002 Genome Biol. 3(8):reviews 3011.1-3011.6; Fearon et al., 1996 Science 272:50; Medzhitov et al., 1997 Curr. Opin. Immunol. 9:4; Luster 2002 Curr. Opin. Immunol. 14:129; Lien et al. 2003 Nat. Immunol. 4:1162; Medzhitov, 2001 Nat. Rev. Immunol. 1:135; Takeda et al., 2003 Ann Rev Immunol. 21:335; Takeda et al. 2005 Int. Immunol. 17:1; Kaisho et al., 2004 Microbes Infect. 6:1388; Datta et al., 2003 J. Immunol. 170:4102).

Induction of TLR-mediated signal transduction to potentiate the initiation of immune responses via the innate immune system may be effected by TLR agonists (i.e., a TLR ligand), which engage cell surface TLR. For example, lipopolysaccharide (LPS) may be a TLR agonist through TLR2 or TLR4 (Tsan et al., 2004 J. Leuk. Biol. 76:514; Tsan et al., 2004 Am. J. Physiol. Cell Phsiol. 286:C739; Lin et al., 2005 Shock 24:206); poly(inosine-cytidine) (polyl:C) may be a TLR agonist through TLR3 (Salem et al., 2006 Vaccine 24:5119); CpG sequences (oligodeoxynucleotides containing unmethylated cytosine-guanosine or “CpG” dinucleotide motifs, e.g., CpG 7909, Cooper et al., 2005 AIDS 19:1473; CpG 10101 Bayes et al. Methods Find Exp Clin Pharmacol 27:193; Vollmer et al. Expert Opinion on Biological Therapy 5:673; Vollmer et al., 2004 Antimicrob. Agents Chemother. 48:2314; Deng et al., 2004 J. Immunol. 173:5148) may be TLR agonists through TLR9 (Andaloussi et a., 2006 Glia 54:526; Chen et al., 2006 J. Immunol. 177:2373); peptidoglycans may be TLR2 and/or TLR6 agonists (Soboll et al., 2006 Biol. Reprod. 75:131; Nakao et al., 2005 J. Immunol. 174:1566); 3M003 (4-Amino-2-(ethoxymethyl)-6,7,8,9-tetrahydro-α,α-dimethyl-1H-imidazo[4,5-c]quinoline-1-ethanol hydrate, Mol. Wt. 318 Da from 3M Pharmaceuticals, St. Paul, Minn., which is also a source of the related compounds 3M001 and 3M002; Gorden et al., 2005 J. Immunol. 174:1259) may be a TLR7 agonist (Johansen 2005 Clin. Exp. Allerg. 35:1591) and/or a TLR8 agonist (Johansen 2005); flagellin may be a TLR % agonist (Feuillet et al., 2006 Proc. Nat. Acad. Sci. USA 103:12487); a profilin may be a TLR11 agonist (Hedhli et al., 2009, Vaccine, 27(16):2274-87); a lipopeptide may be a TLR1, TLR2, and/or TLR6 agonist (Gao et al., 2013, Vaccine, 31(26):2796-803); and hepatitis C antigens may act as TLR agonists through TLR7 and/or TLR9 (Lee et al., 2006 Proc. Nat. Acad. Sci. USA 103:1828; Horsmans et al., 2005 Hepatol. 42:724). Other TLR agonists are known (e.g., Schirmbeck et al., 2003 J. Immunol. 171:5198) and may be used according to certain of the presently described implementations.

In some implementations, the adjuvant is a TLR4 agonist. In some implementations, the TLR4 agonist is a glucopyranosyl lipid adjuvant (GLA), such as those described in US 2007/021017, U.S. Pat. No. 7,661,522, WO 2010/141861, and U.S. Pat. No. 8,722,064. In some implementations, GLA-AF is used. As used herein GLA-AF is a Toll-like receptor 4 agonist glucopyranosyl lipid adjuvant-aqueous nanosuspension (GLA-AF) and consists of the synthetic TLR4 agonist glucopyranosyl lipid adjuvant (GLA) formulated in an aqueous nanosuspension (AF).

In some implementations herein, the adjuvant is a cytokine adjuvant. One or more cytokines can be suitable as an adjuvant alone or in a combination with one or more additional adjuvants in a composition described herein. Suitable cytokines include an interferon (IFN), an interleukin (IL), a chemokine, a colony-stimulating factor, or a tumor necrosis factor. In some implementations, the interferon is a Type I IFN, a Type II IFN, or a Type III IFN. In some implementations, the interferon is IFN-alpha, IFN-beta, IFN-gamma, or IFN-lamda and subtypes from among these (e.g., IFN-lamda, IFN-lamda2, and IFN-lamda3). In some implementations, the cytokine is an interleukin. Non-limiting examples of interleukins that can be used as an adjuvant in a composition described herein include IL-1, IL-2, IL-3, IL-4, IL-5, IL-6, IL-7, IL-8, IL-9, IL-10, IL-11, IL-12, IL-13, IL-15, IL-17, IL-18, IL-19, IL-20, IL-21, IL-22, IL-23, IL-24, IL-25, IL-26, IL-27, IL-28, IL-29, IL-30, IL-31, IL-32, IL-33, IL-35, and IL-36. In some implementations, the cytokine is a chemokine. In some implementations, the chemokine is a CC chemokine, a CXC chemokine, a C chemokine, or a CX3C chemokine. Non-limiting examples of CC chemokines that can be used as an adjuvant in a composition described herein include CCL1, CCL2, CCL3, CCL4, CCL5, CCL6, CCL7, CCL7, CCL8, CCL9, CCL10, CCL11, CCL12, CCL13, CCL14, CCL15, CCL16, CCL17, CCL18, CCL19, CCL20, CCL21, CCL22, CCL23, CCL24, CCL25, CCL26, CCL27, and CCL28.

In some implementations, the cytokine is a colony-stimulating factor. In some implementations, the colony-stimulatory factor is granulocyte macrophage colony-stimulating factor (GM-CSF), granulocyte colony-stimulating factor (G-CSF), or macrophage colony-stimulating factor (M-CSF). In some implementations, the cytokine is a tumor necrosis factor. Non-limiting examples of a tumor necrosis factor (TNF) family protein that can be used as an adjuvant in a composition described herein include TNF-alpha and 4-1BBL.

For example, and by way of background (see, e.g., U.S. Pat. No. 6,544,518) immunostimulatory oligonucleotides containing unmethylated CpG dinucleotides (“CpG”) are known as being adjuvants when administered by both systemic and mucosal routes (WO 96/02555, EP 468520, Davis et al., J. Immunol, 1998. 160(2):870-876; McCluskie and Davis, J. Immunol., 1998, 161(9):4463-6). CpG is an abbreviation for cytosine-guanosine dinucleotide motifs present in DNA. The central role of the CG motif in immunostimulation was elucidated by Krieg, Nature 374, p 546 1995.

Detailed analysis has shown that the CG motif has to be in a certain sequence context and that such sequences are common in bacterial DNA but are rare in vertebrate DNA. The immunostimulatory sequence is often: Purine, Purine, C, G, pyrimidine, pyrimidine; wherein the dinucleotide CG motif is not methylated, but other unmethylated CpG sequences are known to be immunostimulatory and may also be used. CpG when formulated into vaccines, may be administered in free solution together with free antigen (WO 96/02555; McCluskie and Davis, supra) or covalently conjugated to an antigen (PCT Publication No. WO 1998/16247) or formulated with a carrier such as aluminum hydroxide (e.g., Davis et al. supra, Brazolot-Millan et al., Proc. Natl. Acad. Sci., USA, 1998, 95(26), 15553-8).

In some implementations, the oligonucleotides for use as an adjuvant contain two or more dinucleotide CpG motifs separated by at least three, including but not limited to, at least six or more nucleotides. The oligonucleotides are typically deoxynucleotides. In some implementations the internucleotide in the oligonucleotide is phosphorodithioate, including but not limited to, a phosphorothioate bond, although phosphodiester and other internucleotide bonds including oligonucleotides with mixed internucleotide linkages are also suitable types of bonds. Methods for producing phosphorothioate oligonucleotides or phosphorodithioate are described in U.S. Pat. Nos. 5,666,153, 5,278,302 and WO 1995/26204.

Examples of oligonucleotides have sequences that are disclosed in the following publications and can be used for certain herein disclosed implementations the sequences contain phosphorothioate modified internucleotide linkages: (1) CPG 7909: Cooper et al., “CPG 7909 adjuvant improves hepatitis B virus vaccine seroprotection in antiretroviral-treated HIV-infected adults.” AIDS, 2005 Sep. 23; 19(14):1473-9; (2) CpG 10101: Bayes et al., “Gateways to clinical trials.” Methods Find. Exp. Clin. Pharmacol. 2005 April; 27(3):193-219; and (3) Vollmer J., “Progress in drug development of immunostimulatory CpG oligodeoxynucleotide ligands for TLR9.” Expert Opinion on Biological Therapy. 2005 May; 5(5): 673-682.

Alternative CpG oligonucleotides may comprise variants of the sequences described in the above-cited publications that differ in that they have inconsequential nucleotide sequence substitutions, insertions, deletions, and/or additions thereto. The CpG oligonucleotides may be synthesized by any method known in the art (e.g., EP 468520). Conveniently, such oligonucleotides may be synthesized utilizing an automated synthesizer. The oligonucleotides are typically deoxynucleotides. In some implementations, the internucleotide bond in the oligonucleotide is a phosphorodithioate or phosphorothioate bond, although phosphodiesters are also within the scope of the presently contemplated implementations. Oligonucleotides comprising different internucleotide linkages are also contemplated, e.g., mixed phosphorothioate phosphodiesters. Other internucleotide bonds that stabilize the oligonucleotide may also be used.

In another implementation, the adjuvant is an attenuated lipid A derivative (ALD). ALDs are lipid A-like molecules that have been altered or constructed so that the molecule displays lesser or different of the adverse effects of lipid A. These adverse effects include pyrogenicity, local Shwarzman reactivity and toxicity as evaluated in the chick embryo 50% lethal dose assay (CELD50) ALDs include monophosphoryl lipid A (MLA) and 3-deacylated monophosphoryl lipid A (3D-MLA). MLA and 3D-MLA are known and need not be described in detail herein. See for example U.S. Pat. No. 4,436,727 issued Mar. 13, 1984, assigned to Ribi ImmunoChem Research, Inc., which discloses monophosphoryl lipid A and its manufacture. U.S. Pat. No. 4,912,094 and reexamination certificate B1 U.S. Pat. No. 4,912,094 to Myers, et al., also assigned to Ribi ImmunoChem Research, Inc., embodies 3-deacylated monophosphoryl lipid A and a method for its manufacture.

In some implementations, response modifiers such as imidazoquinoline and other immune response modifiers known in the art and may also be included as adjuvants in certain presently disclosed implementations. Certain imidazoquinoline immune response modifiers include, by way of non-limiting example, resiquimod (R848), imiquimod and gardiquimod (Hemmi et al., 2002 Nat. Immunol. 3:196; Gibson et al., 2002 Cell. Immunol. 218:74; Gorden et al., 2005 J. Immunol. 174:1259); these and other imidazoquinoline immune response modifiers may, under appropriate conditions, also have TLR agonist activity as described herein. Other immune response modifiers are the nucleic acid-based double stem-loop immune modifiers (dSLIM). Specific examples of dSLIM that are contemplated for use in certain of the presently disclosed implementations can be found in Schmidt et al., 2006 Allergy 61:56; Weihrauch et al. 2005 Clin Cancer Res. 11(16):5993-6001; Modern Biopharmaceuticals, J. Knablein (Editor). John Wiley & Sons, Dec. 6, 2005. (dSLIM discussed on pages 183 to about 200).

In some implementations, an adjuvant used in a composition described herein is a polysaccharide derived from bacteria or plants. Non-limiting examples of polysaccharide-based adjuvants that can be used alone or in combination with one or more additional adjuvant in a composition described herein include glucans (e.g., beta glucans), dextrans (e.g., sulfated and diethylaminoethyl-dextrans), glucomannans, galactomannans, levans, xylans, fructans (e.g., inulin), chitosan, endotoxins (e.g., lipopolysaccharide), biobran MGN-3, polysaccharides from Actinidia eriantha, eldexomer, and variations thereof.

In some implementations, an adjuvant used in a composition described herein is a proteosome or subunit thereof. In some implementations, an adjuvant used in a composition described herein comprises identical or different antigenic peptide sequences assembled around a lysine core.

In some implementations, an adjuvant used in a composition described herein is a toxin (e.g., a bacterial toxin). In some implementations, the toxin is from one or more bacteria selected from the group consisting of Escherichia coli, Vibrio cholera, Bordetella pertussis, and Bordetella parapertussis.

In some implementations, an adjuvant used in a composition described herein (e.g., RIG-I agonist/adjuvant formulation) is a delivery adjuvant. A delivery adjuvant can serve as an adjuvant and/or can deliver an antigen. Non-limiting examples of an adjuvant that can be used alone or in combination with one or more additional adjuvant in a composition described herein includes mineral salts (e.g., calcium phosphate), emulsions (e.g., squalene in water), liposomes (e.g., DPPC:cholesterol liposomes), virosomes (e.g., immunopotentiating reconstituted influenza virosomes), and microspheres.

Other adjuvants for use according to certain herein disclosed implementations include a block co-polymer or biodegradable polymer, which refers to a class of polymeric compounds with which those in the relevant art will be familiar. Examples of a block co-polymer or biodegradable polymer that may be included in a composition described herein include Pluronic® L121 (BASF Corp., Mount Olive, N.J.; see, e.g., Yeh et al., 1996 Pharm. Res. 13:1693; U.S. Pat. No. 5,565,209), CRL1005 (e.g., Triozzi et al., 1997 Clin Canc. Res. 3:2355), poly(lactic-co-glycolic acid) (PLGA), poly(lactic acid) (PLA), poly-(D,L-lactide-co-glycolide) (PLG), and polyl:C. (See, e.g., Powell and Newman, “Vaccine design—The Subunit and Adjuvant Approach”, 1995, Plenum Press, New York).

In some implementations, an adjuvant used in a composition described herein (e.g., RIG-I agonist/adjuvant formulation) is an organic adjuvant. Organic adjuvants can be adjuvants that are derived from living organisms or chemically contain carbon. In some implementation, the adjuvant is a peptide derived from a microbial cell wall (e.g., muramyl dipeptide and variants thereof). In some implementations, the adjuvant is trehalose 6,6′-dimycolate or variants thereof. See Schweneker et al., 2013, Immunobiology, 218(4):664-73. In some implementations, the adjuvant is stearyl tyrosine.

Saponins and saponin mimetics, including QS21 and structurally related compounds conferring similar effects and referred to herein as QS21 mimetics (see, e.g., U.S. Pat. No. 5,057,540; EP 0 362 279 B1; WO 95/17210), plant alkaloids such as tomatine, detergents such as (but not limited to) saponin, polysorbate 80, Span 85 and stearyl tyrosine, an imidazoquinoline immune response modifier, and a double stem-loop immune modifier (dSLIM, e.g., Weeratna et al., 2005 Vaccine 23:5263) may be used as an adjuvant according to certain of the presently described implementations.

In some implementations, the adjuvant used in a composition described herein is a saponin or a saponin mimetic. Detergents including saponins are taught in, e.g., U.S. Pat. No. 6,544,518; Lacaille-Dubois, M and Wagner H. (1996 Phytomedicine 2:363-386), U.S. Pat. No. 5,057,540, Kensil, Crit Rev Ther Drug Carrier Syst, 1996, 12 (1-2):1-55, and EP 0 362 279 B1. Particulate structures, termed Immune Stimulating Complexes (ISCOMS), comprising fractions of Quil A (saponin) are hemolytic and have been used in the manufacture of vaccines (Morein, B., EP 0 109 942 B1). These structures have been reported to have adjuvant activity (EP 0 109 942 B1; WO 96/11711). The hemolytic saponins QS21 and QS17 (HPLC purified fractions of Quil A) have been described as potent systemic adjuvants, and the method of their production is disclosed in U.S. Pat. No. 5,057,540 and EP 0 362 279 B1. QS21 may comprise an HPLC purified non-toxic fraction derived from the bark of Quillaja saponaria Molina. The production of QS21 is disclosed in U.S. Pat. No. 5,057,540 (See also U.S. Pat. Nos. 6,936,255, 7,029,678 and 6,932,972.). Also described in these references is the use of QS7 (a non-haemolytic fraction of Quil-A) which acts as a potent adjuvant for systemic vaccines. Use of QS21 is further described in Kensil et al. (1991. J. Immunology 146:431-437). Combinations of QS21 and polysorbate or cyclodextrin are also known (WO 99/10008). Particulate adjuvant systems comprising fractions of QuilA, such as QS21 and QS7 are described in WO 96/33739 and WO 96/11711. Other saponins which have been used in systemic vaccination studies include those derived from other plant species such as Gypsophila and Saponaria (Bomford et al., Vaccine, 10(9):572-577, 1992).

In some implementations, the adjuvant is an “immunostimulatory complex” known as ISCOMS (e.g., U.S. Pat. Nos. 6,869,607, 6,846,489, 6,027,732, 4,981,684), including saponin-derived ISCOMATRIX®, which is commercially available, for example, from Iscotec (Stockholm, Sweden) and CSL Ltd. (Parkville, Victoria, Australia).

Escin is another detergent related to the saponins for use in the adjuvant compositions of the implementations herein disclosed. Escin is described in the Merck index (12th Ed.: entry 3737) as a mixture of saponin occurring in the seed of the horse chestnut tree, Aesculus hippocastanum. Its isolation is described by chromatography and purification (Fiedler, Arzneimittel-Forsch. 4, 213 (1953)), and by ion-exchange resins (Erbring et al., U.S. Pat. No. 3,238,190). Fractions of escin (also known as aescin) have been purified and shown to be biologically active (Yoshikawa M, et al. (Chem Pharm Bull (Tokyo) 1996 August; 44(8): 1454-1464)).

Digitonin is another detergent, also described in the Merck index (12th Ed., entry 3204) as a saponin. It is derived from the seeds of Digitalis purpurea and purified according to the procedure described by Gisvold et al., J. Am. Pharm. Assoc., 1934, 23, 664; and Rubenstroth-Bauer, Physiol. Chem., 1955, 301, 621.

In some implementations, an adjuvant used in a composition described herein (e.g., RIG-I agonist/adjuvant formulation) is an inorganic adjuvant. Inorganic adjuvants can be adjuvants that are generally not carbon-based such as, for example, mineral salts, emulsions, and calcium phosphates. Mineral salts adjuvants contemplated herein include, but are not limited to, aluminum-based compounds such as aluminum phosphate and aluminum hydroxide. As used herein, calcium phosphate adjuvants include, but are not limited to, calcium ions (Ca²⁺) together with orthophosphates (PO₄³⁻), metaphosphates (PO³⁻), or pyrophosphates (P₂O₇⁴).

### Excipients

“Excipients” as used herein refers to substances other than the pharmacologically or immunologically active agents. Excipients are included in the manufacturing process, or fill-finish process for storage or shipment of a pharmacologically active drug or immunologically agent. Excipients are substances besides agonists and adjuvants that are included in any of the formulations of this disclosure. Excipients may be bulking agents, buffering agents, emulsifiers, or solubilizing agents. Lyophilization excipients refer to substances that are included in a lyophilization process to contribute to the form or formulation of a suitable cake structure.

Excipients suitable for formulations of RIG-I agonists and adjuvants are known in the art (See, e.g. Bahetia et. al., 2010: J. Excipients and Food Chem.:1 (1)41-54, Grabenstein J D. ImmunoFacts: Vaccines and Immunologic Drugs—2012 (37th revision). St Louis, Mo.: Wolters Kluwer Health, 2011 and, by Vaccine) and include lyoprotectants, cryoprotectants, cake-forming excipients, cake-forming bulking agents, bulking agents, buffering agents, solubilizing agents, isotonicity agents, tonicifying agents, surfactants, emulsifiers, antimicrobial agents, and collapse temperature modifiers.

In some implementations, excipients approved for vaccines can be used in the formulation herein and can be found via the Centers for Disease Control (see the worldwide web at cdc.gov/vaccines/pubs/pinkbook/downloads/appendices/b/excipient-table-2.pdf, last visited Nov. 3, 2020, “Vaccine Excipient Summary. Excipients Included in U.S. Vaccines, by Vaccine”). Approved excipients include, but are not limited to, sucrose, D-mannose, D-fructose, dextrose, potassium phosphate, plasdone C, anhydrous lactose, micro crystalline cellulose, polacrilin potassium, magnesium stearate, cellulose acetate phthalate, alcohol, acetone, castor oil, FD&C Yellow #6 aluminum lake dye, human serum albumin, fetal bovine serum, sodium bicarbonate, human-diploid fibroblast cell cultures (WI-38), Dulbecco's Modified Eagle's Medium, aluminum hydroxide, benzethonium chloride, formaldehyde, gluteraldehyde, amino acids, vitamins, inorganic salts, sugars, glycerin, asparagine, citric acid, potassium phosphate, magnesium sulfate, iron ammonium citrate, lactose, aluminum potassium sulfate, aluminum hydroxyphosphate, potassium aluminum sulfate, peptone, bovine extract, thimerosal (trace), modified Mueller and Miller medium, beta-propiolactone, thimerosol (multi-dose vials only), monobasic sodium phosphate, dibasic sodium phosphate, monobasic potassium phosphate, potassium chloride, potassium glutamate, calcium chloride, sodium taurodeoxycholate, neomycin sulfate, polymyxin B, egg protein, lactalbumin hydrolysate, and neomycin sulfate.

In some implementations, an excipient is a substance added to an emulsion formulation prior to lyophilization which yields a cake following lyophilization, including cake-forming excipients or cake-forming bulking agents. In some implementations, a cake-forming excipient is a substance added to an emulsion formulation prior to lyophilization which yields a cake following lyophilization. Upon reconstitution of the lyophilized cake a stable emulsion forms. In some implementations, the stable emulsion is an oil-in-water stable emulsion that is suitable for the delivery of a RIG-I agonist. In some implementations, cake-forming excipients are those substances that do not disrupt an emulsion upon reconstitution of the cake. In some implementations the agents useful as cake-forming excipients also referred to as bulking agents, include sugars/saccharides or sugars/saccharides in combination with sugar alcohols. In some implementations disclosed herein, the sugars/saccharides or sugars/saccharides in combination with sugar alcohols are useful as bulking agents or cake-forming excipients include. These include, but are not limited to, trehalose, dextrose, lactose, maltose, sucrose, raffinose, mannose, stachyose, fructose, lactulose, glucose, and optionally glycerol, sorbitol, and/or mannitol.

In some implementations, the excipient is a saccharide selected from the group consisting of trehalose, dextrose, lactose, maltose, sucrose, raffinose, mannose, stachyose, fructose, and lactulose. In some implementations, the excipient is a combination of mannitol or sorbitol and a saccharide.

In some implementations, an excipient may be a buffering agent. Buffering agents useful as excipients include Tris acetate, Tris base, Tris HCl, Ammonium phosphate, Citric Acid, Sodium Citrate, Potassium citrate, Tartic Acid, Sodium Phosphate, Zinc Chloride, Arginine, and Histidine. In some implementations buffering agents include pH adjusting agents such as hydrochloric acid, sodium hydroxide, and meglumine.

In some implementations, an excipient may be a solubilizing agent. In some implementations, suitable solubilizing agents include complexing excipients such as ethylenediaminetetraacetic acid (EDTA), Alpha cyclodextrin, Hydroxypropyl-beta-cyclodextrin (HP-beta-CD). Surfactants may also be included as solubilizing excipients including polysorbate 80 and Tween. Other Co-Solvents known in the art as solubilizing agents may be used and include tert-butyl alcohol, isopropyl alcohol, dichloromethane, ethanol, and acetone.

In some implementations, an excipient may be a tonicitying agent, a collapse temperature modifier, and antimicrobial agent, an isotonicity agent, a surfactant, or an emulsifier. Tonicifying agents suitable for use as excipients include glycerol, sodium chloride, sucrose, mannitol, and dextrose. Collapse temperature modifiers include dextran, hydroxyethyl starch, ficoll, and gelatin. Antimicrobial agents include benzyl alcohol, phenol, m-cresol, methyl paraben, ethyl paraben, and thimerosol. A suitable isotonicity agent is glycerol. A suitable surfactant is pluronic F68. Suitable emulsifiers are 1,2-dimyristoyl-sn-glycero-3-phosphocholine (DMPC) and lecithin.

### Formulations

As used herein, “formulation” includes “pharmaceutical formulation,” “co-formulation,” and “single vial formulation.” A formulation at least one RIG-I agonist and optionally one or more adjuvants. Formulations are known to those skilled in the art and include but are not limited to injectable formulations and dispersion of the active agent in a medium that is insoluble in physiologic fluids or where the release of the antigen and/or adjuvant is released after degradation of the formulation due to mechanical, chemical, or enzymatic activity. Formulations may also include one or more excipients.

Production of the RIG-I agonist/adjuvant formulations can be via any method known in the art. In some implementations, the formulation of the RIG-I agonist and the adjuvant are produced in a single vial for administration. In some implementations, a formulation is diluted with an excipient (e.g., dextrose water) and then combined with other excipients (e.g., polysorbate 80 and lecithin) before administration. The method of admixture and the order of admixture of the components of the formulation may depend upon the adjuvant being used. In some implementations, the RIG-I agonist is diluted to a concentration of more than 1× (e.g., 2× or 3×) and then diluted into the adjuvant to an effective concentration (1×). In some implementations, the final concentration of the adjuvant in the formulation is an effective concentration. In some implementations, the RIG-I agonist and the adjuvant are admixed with an emulsion together or separately to create a formulation of the RIG-I agonist and adjuvant. In some implementations, one or more excipients are then admixed with the formulation in a single vial. In some implementations, the excipients can be added in any order with the RIG-I agonist and the adjuvant.

In some implementations, the adjuvant is a squalene emulsion (SE) and a concentrated amount of the RIG-I agonist is admixed with the squalene emulsion to dilute the RIG-I adjuvant to a 1× concentration (an effective concentration) and the squalene emulsion is diluted to an effective concentration.

In some implementations, the RIG-I agonist is admixed with a nanostructured lipid carrier (NLC) adjuvant. In some implementations, the RIG-I agonist RNA molecule is complexed with a nanostructured lipid carrier (NLC) by association with the cationic surface. The association of the RNA molecule with the NLC surface may be a non-covalent or reversible covalent interaction. In some implementations, the NLC is formulated and then the RIG-I agonist is mixed in using a single vial.

In some implementations, the RIG-I agonist and adjuvant formulation is lyophilized. In some implementations, the RIG-I agonist and adjuvant is provided in a single vial.

While a formulation or a formulation in a single vial is envisioned, other implementations may include a separate formulation of the RIG-I agonist and a separate formulation of the adjuvant that are admixed prior to administration. In other implementations, the two separate formulations are administered at the same time to the subject but are formulated separately. In other implementations, the two separate formulations are administered to the same subject within at least one week, including within at least 6 days, 5 days, 4 days, 3 days, 2 days, 1 day, 12 hours, 11 hours, 10 hours, 9 hours, 8 hours, 7 hours, 6 hours, 5 hours, 4 hours, 3 hours, 1 hour, 30 minutes, 15 minutes, 5 minutes, or less. In some implementations, the RIG-I agonist formulation is administered first and the adjuvant formulation is administered second within 1 week of administration of the RIG-I agonist formulation.

### Compositions of RIG-I Agonists and Adjuvants

Compositions comprising the RIG-I agonist/adjuvant formulations provided herein may be referred to as formulations, pharmaceutical formulations, compositions, and pharmaceutical compositions. The compositions comprising the RIG-I agonist/adjuvant can optionally further comprise a pharmaceutically acceptable carrier, excipient, and/or diluent. The compositions described may be administered to a subject as a therapeutic. A “subject” is a patient, individual, person, or animal that receives a RIG-I agonist/adjuvant formulation. Animals include, but are not limited to, mammals. Mammals include, but are not limited to humans, farm animals, sport animals, domestic animals (e.g., cats, dogs, horses), primates, mice, and rats.

The compositions described herein can be used in the treatment of a cancerous or precancerous condition, which may be diagnosed or not. In some implementations, the cancerous or precancerous condition is a solid tumor. The term “solid tumor” as used herein applies to an abnormal mass of tissue that usually does not contain cysts or liquid areas and can arise in any part of the body. Solid tumors may be benign (not cancerous) or malignant (cancerous). Most kinds of cancer other than leukemias can form solid tumors. In general, solid tumors are well-defined as opposed to diffuse masses of tissue and typically have a three-dimensional shape. The cancerous or precancerous condition can occur in any organ or body part, including without limitation, anus, bile duct, bone marrow, brain, breast, cervix, colon, duodenum, esophagus, gallbladder, head and neck, ileum, jejunum, kidney, larynx, liver, lung, mouth, ovary, pancreas, pelvis, penis, pituitary, prostate, rectum, skin, stomach, testes, thyroid, urinary bladder, uterus, and vagina.

In some implementations, the pharmaceutical compositions comprising a RIG-I agonist/adjuvant are administered to a subject in a therapeutically effective amount. The term “effective amount” or “therapeutically effective amount” refers to an amount that is sufficient to achieve or at least partially achieve the desired effect, e.g., sufficient to generate the desired therapeutic response. An effective amount of a composition is administered in an “effective regime.” The term “effective regime” refers to a combination of an amount of the composition being administered and dosage frequency adequate to accomplish the desired effect. In some implementations, the desired effect for the RIG-I agonist differs from the desired effect for the adjuvant. In some implementations, the desired effect is measured for the combination of the RIG-I agonist and the adjuvant and the combined effect is measured or analyzed.

The dosage to achieve an “effective amount” of the RIG-I agonist and/or the adjuvant is an amount that is sufficient to achieve or at least partially achieve the desired effect. Actual dosage levels may be varied so as to obtain an amount that is effective to achieve the desired response for a particular subject, composition, and mode of administration, without being toxic to the subject. The selected dosage level may depend upon a variety of pharmacokinetic factors in combination with the particular compositions employed, the age, sex, weight, condition, general health and prior medical history of the subject being treated, and similar factors that are 6 well-known in the medical arts. In some therapeutic implementations, a dosage of about 0.5 ng to about 100 ng of a therapeutic pharmaceutical composition is administered. It will be evident to those skilled in the art that the number and frequency of administrations will be dependent upon the response of the subject.

“Pharmaceutically acceptable carriers” for therapeutic use are well known in the pharmaceutical art and are described, for example, in Remington's Pharmaceutical Sciences, Mack Publishing Co. (A. R. Gennaro edit. 1985). For example, sterile saline and phosphate-buffered saline at physiological pH may be used. Preservatives, stabilizers, dyes and even flavoring agents may be provided in the pharmaceutical composition. For example, sodium benzoate, sorbic acid, and esters of alpha-hydroxybenzoic acid may be added as preservatives. In addition, antioxidants and suspending agents may be used.

The pharmaceutical compositions may be in any form which allows for the composition to be administered to a subject. For example, the composition may be in the form of a solid, liquid or gas (aerosol). Typical routes of administration include, without limitation, intratumoral, intravenous, subcutaneous, intradermal, intraperitoneal, intracranial, and intrathecal. The term parenteral as used herein includes iontophoretic, sonophoretic, thermal, transdermal administration and also subcutaneous injections, intravenous, intramuscular, intrasternal, intracavernous, intrathecal, intrameatal, intraurethral injection or infusion techniques. In some implementations, a composition as described herein (including vaccine and pharmaceutical compositions) is administered intradermally by a technique selected from iontophoresis, microcavitation, sonophoresis, jet injection, or microneedles. In some implementations, a composition as described herein is administered intradermally using the microneedle device manufactured by NanoPass Technologies Ltd., Nes Ziona, Israel, e.g., MicronJet600 (see, e.g., U.S. Pat. Nos. 6,533,949 and 7,998,119 and Yotam, et al., Human vaccines & immunotherapeutics 11(4): 991-997 (2015).

The pharmaceutical composition can be formulated to allow the active ingredients contained therein to be bioavailable upon administration of the composition to a subject. Compositions that will be administered to a subject take the form of one or more dosage units.

The composition may be in the form of a liquid, e.g., a solution, emulsion, or suspension. In a composition intended to be administered by injection by needle and syringe or needle-free jet injection, one or more of a surfactant, preservative, wetting agent, dispersing agent, suspending agent, buffer, stabilizer, and isotonic agent may be included.

A liquid pharmaceutical composition as used herein, whether in the form of a solution, suspension or other like form, may include one or more of the following carriers or excipients: sterile diluents such as water for injection, saline solution, including but not limited to, physiological saline, Ringer's solution, isotonic sodium chloride, fixed oils such as squalene, squalane, mineral oil, a mannide monooleate, cholesterol, and/or synthetic mono or diglycerides which may serve as the solvent or suspending medium, polyethylene glycols, glycerin, propylene glycol or other solvents; antibacterial agents such as benzyl alcohol or methylparaben; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. In another implementation, a composition of the present disclosure is formulated in a manner which can be aerosolized. The composition may be intended for rectal administration, in the form, e.g., of a suppository which can melt in the rectum and release the drug.

It may also be desirable to include other components in a pharmaceutical composition, such as delivery vehicles including but not limited to aluminum salts, water-in-oil emulsions, biodegradable oil vehicles, oil-in-water emulsions, biodegradable microcapsules, and liposomes. Examples of additional immunostimulatory substances (co-adjuvants) for use in such vehicles are also described above and may include N-acetylmuramyl-L-alanine-D-isoglutamine (MDP), glucan, IL-12, GM-CSF, gamma interferon, and IL-12.

While any suitable carrier known to those of ordinary skill in the art may be employed in the pharmaceutical compositions of the present disclosure, the type of carrier will vary depending on the mode of administration and whether a sustained release is desired. For parenteral administration, such as subcutaneous injection, the carrier can comprise water, saline, alcohol, a fat, a wax, or a buffer.

Pharmaceutical compositions may also contain diluents such as buffers, antioxidants such as ascorbic acid, polypeptides, proteins, amino acids, carbohydrates including glucose, sucrose or dextrins, chelating agents such as EDTA, glutathione, and other stabilizers and excipients. Neutral buffered saline or saline mixed with nonspecific serum albumins are examples of appropriate diluents. For example, a product may be formulated as a lyophilizate using appropriate excipient solutions (e.g., sucrose) as diluents.

Optionally, to control tonicity, the composition may comprise a physiological salt, such as a sodium salt. Other salts that may be present include potassium chloride, potassium dihydrogen phosphate, disodium phosphate, magnesium chloride, calcium chloride, etc. Non-ionic tonicifying agents can also be used to control tonicity. Monosaccharides classified as aldoses such as glucose, mannose, arabinose, and ribose, as well as those classified as ketoses such as fructose, sorbose, and xylulose can be used as non-ionic tonicifying agents in the presently disclosed compositions. Disaccharides such as sucrose, maltose, trehalose, and lactose can also be used. In addition, alditols (acyclic polyhydroxy alcohols, also referred to as sugar alcohols) such as glycerol, mannitol, xylitol, and sorbitol are non-ionic tonicifying agents useful in the presently disclosed compositions. If the composition is formulated for parenteral administration, the osmolarity of the composition may be made the same as normal physiological fluids, preventing post-administration consequences, such as post-administration swelling or rapid absorption of the composition. Optionally, the composition may be formulated with cryoprotectants comprising trehalose, sucrose, mannitol, sorbitol, Avicel PHI 02 (microcrystalline cellulose), Avicel RC591 (mixture of microcrystalline cellulose and sodium carboxymethyl cellulose), Mircrocelac® (mixture of lactose and Avicel), or a combination thereof. Optionally, the composition may be formulated with a preservative agent such as, for example, Hydrolite 5.

### Administration of RIG-I Agonists in Combination with Adjuvants

The RIG-I agonists/adjuvant formulations provided in this disclosure have use in enhancing or eliciting in a subject or in cell culture activation of the RIG-I receptor and associated immune response. Provided herein are methods for treating cancer by slowing tumor growth in a subject using the formulations of one or more RIG-I agonists and optionally one or more adjuvants. The methods may incorporate activation of the RIG-I receptor via the RIG-I agonist and then simulate an immune response in a subject via the adjuvant. The method may further comprise a step of diluting or reconstituting the RIG-I agonist/adjuvant formulation before administration.

In some implementations, a RIG-I agonist/adjuvant emulsion may be administered to a subject to stimulate an immune response, e.g., a non-specific immune response or an antigen-specific immune response, for the purpose of treating or preventing cancer in a subject. In some implementations, the cancer is a solid tumor. In some implementations, the solid tumor is selected from liver, head and neck, pancreatic, and melanoma. In some other implementations, the pharmaceutical composition is a formulation that comprises the compositions described herein in combination with a pharmaceutically acceptable carrier, excipient, or diluent. Illustrative carriers are usually nontoxic to recipients at the dosages and concentrations employed. However, it is contemplated that the RIG-I agonist emulsion and the adjuvant may be administered separately.

A subject may be afflicted with cancer, such as breast cancer, or may be free of detectable cancer. In example formulations provided herein, about 0.1 ng to about 120 ng, about 4 ng to about 120 ng, about 20 ng to about 1 ng, about 90 ng to about 110 ng, or about 100 ng of the RIG-I agonist can be administered in a single dose. The amount of RIG-I agonist administered in a single dose may be any of about, 0.75, 1.0, 1.5, 2.0, 2.5, 3.0, 3.5, 4.0, 5.0, 10.0, 15, 20, 25, 30, 35, 40, 45, 50, 55, 60, 65, 70, 75, 80, 85, 90, 95, 100, 105, 110, 115, and 120 ng.

Administration of the RIG-I agonist may be performed so that an effective amount of the RIG-I agonist is delivered to the subject. As used herein, “effective amount” of the RIG-I agonist is an amount that induces an immune response in the subject. Inducing an immune response includes, but is not limited to, activating a RIG-I receptor in the subject. One possible result of activating a RIG-I receptor is triggering a signaling cascade that leads to the production of type I IFNs and pro-inflammatory cytokines in the subject. An effective amount may be administered through an “effective regime.” The term “effective regime” refers to a combination of the amount of the composition being administered and dosage frequency adequate to accomplish the desired effect. It will be evident to those skilled in the art that the number and frequency of administrations will be dependent upon the response of the subject. Formulations may achieve therapeutic efficacy after as little as one administration.

### Routes of Administration

There are many suitable routes of administration for the RIG-I agonist/adjuvant formulations described in this disclosure. The routes of administration include, but are not limited to, intravenous, subcutaneous, intracranial, intrathecal, intratumoral and other parenteral routes of administration, including, but not limited to, intramuscular, intraperitoneal, intraspinal, intracerebroventricular, and intraarterial. In some implementations, a formulation is lyophilized and the lyophilized co-formulation is reconstituted prior to administration to a subject. Compositions containing the RIG-I agonist/adjuvant formulations of this disclosure can provide controlled, slow release, or sustained release of the RIG-I agonist and/or adjuvant over a predetermined period of time.

Therapeutic treatments of a subject such as treatment or prevention of solid tumors, or cancer. In some implementations, the cancer is a solid tumor. In some implementations, the solid tumor is selected from a liver, head and neck, pancreatic and melanoma tumor.

### Kits and Pharmaceutical Packs

Also contemplated in certain implementations are kits and pharmaceutical packs comprising RIG-I agonist/adjuvant formulations which may be provided in one or more containers as a liquid or lyophilized. In one implementation all components of a formulation are present together in a single container or vial (i.e., co-formulated). However, the agonist and the adjuvant may be provided in two or more separate containers.

As used herein, “container” includes vessel, vial, ampule, tube, cup, box, bottle, flask, jar, dish, well of a single-well or multi-well apparatus, reservoir, tank, or the like, or other device in which the herein disclosed compositions may be placed, stored and/or transported, and accessed to remove the contents. Typically, such a container may be made of a material that is compatible with the intended use and from which recovery of the container contents can be readily achieved. Examples of such containers include glass and/or plastic sealed or re-sealable tubes and ampules, including those having a rubber septum or other sealing means that is compatible with withdrawal of the contents using a needle and syringe.

In some implementations, the use of the term “vial” means that any appropriate container can be used, including a vial. Such containers may, for instance, be made of glass or a chemically compatible plastic or resin, which may be made of, or may be coated with, a material that permits efficient recovery of material from the container and/or protects the material from, e.g., degradative conditions such as ultraviolet light or temperature extremes, or from the introduction of unwanted contaminants including microbial contaminants. In some implementations, the containers are sterile or sterilizable, and made of materials that will be compatible with any carrier, excipient, solvent, vehicle or the like, such as may be used to suspend or dissolve the compositions and formulations disclosed herein. In some implementations, the containers are RNase free.

### General Techniques

The practice of the present disclosure will employ, unless otherwise indicated, conventional techniques of molecular biology, recombinant DNA, biochemistry, and chemistry, which are within the skill of the art. Such techniques are explained fully in the literature. See, e.g., Molecular Cloning A Laboratory Manual, 2nd Ed., Sambrook et al., ed., Cold Spring Harbor Laboratory Press: (1989); DNA Cloning, Volumes I and II (D. N. Glover ed., 1985); Oligonucleotide Synthesis (M. J. Gait ed., 1984); Mullis et al., U.S. Pat. No. 4,683,195; Nucleic Acid Hybridization (B. D. Hames & S. J. Higgins eds. 1984); B. Perbal, A Practical Guide To Molecular Cloning (1984); the treatise, Methods In Enzymology (Academic Press, Inc., N.Y.); and in Ausubel et al., Current Protocols in Molecular Biology, John Wiley and Sons, Baltimore, Md. (1989).

### EXAMPLES

The following examples demonstrate the efficacy of RIG-I agonists as a therapeutic treatment that can limit tumor growth. RIG-I agonists 3p-hpRNA and pUUC AuK were administered to C57BL/6 mice injected with B16 (melanoma) tumor cells. Examples also test the effect of SE formalization. The materials and methods used in the examples are described below.

Mice. Female C57BL/6 mice (purchased from Charles River Laboratories, Wilmington, Mass.) and were maintained in specific pathogen-free conditions and in accordance with established care animal procedures. Mice entered experiments at 6-8 weeks of age.

B16 melanoma tumor cells. B16 melanoma is a murine tumor cell line used for research as a model for human skin cancers. B16 cells are useful models for the study of metastasis and solid tumor formation. B16 cells were maintained by tissue culture and were prepared for injection during log-phase growth. Cells were collected from tissue culture flasks, washed a minimum of two times in phosphate buffered saline (PBS), counted and then diluted to provide 1×10⁵ cells per 100 μl for injection into mice. Prior to tumor cell inoculation, each mouse was anesthetized by injection of ketamine/xylazine and the abdomen was shaved to remove hair. Other mice were injected with a total volume of 100 μl of the B16 cell suspension subcutaneously on the left side of their abdomens. The day of B16 cell injection is referred to as day 0 of tumor development. Tumors were measured for length, breadth, and protruding height using a digital engineering-grade micrometer.

Treatments. Either of two RIG-I agonists, 3p-hpRNA (InvivoGen, San Diego, Calif.) and pUUC AuK, or a negative agonist control XRNA were diluted in 5% dextrose water to provide 2× concentration. For experiments using a formulated agonist, the 2× concentrations of agonists described above were then diluted 2-fold into a formulation with SE comprising 1% squalene+0.2% polysorbate 80+1.62 mg/mL lecithin. Abdomens were again shaved to remove hair and the mice were either (1) injected through the shaved region of the abdomen with a total volume of 100 μl subcutaneously proximate to the tumor cell injection site on day one of tumor development (i.e., before the tumor B 16 tumor cells had an opportunity to grow to a detectable size) or (2) 100 μl was injected directly into a tumor on day 17 of tumor development.

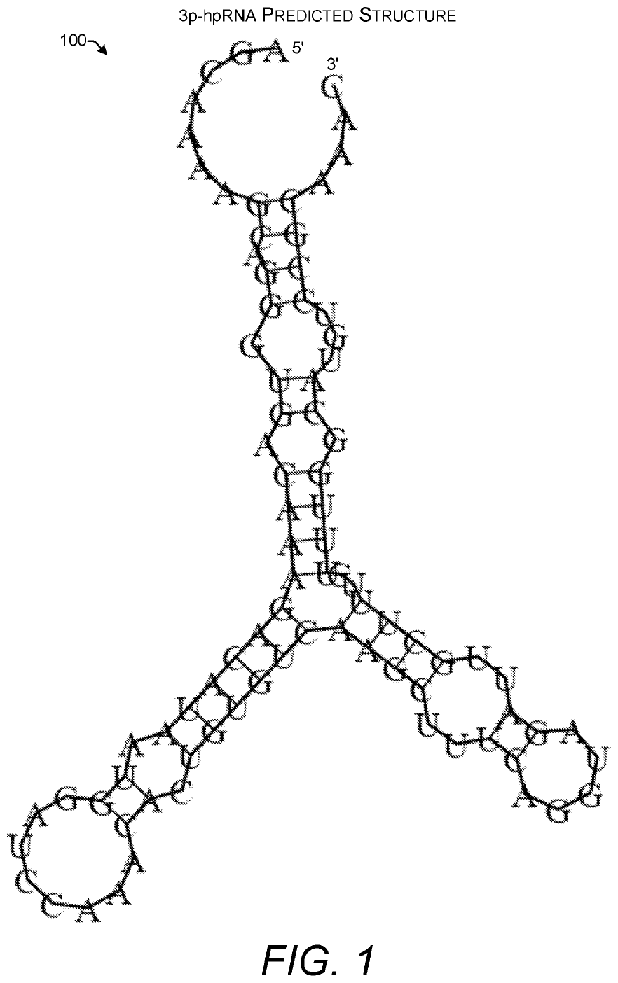

3p-hpRNA. This known RIG-I agonist is a 5′ triphosphate hairpin RNA that was generated by in vitro transcription of a sequence from the influenza A (H1N1) virus, a single-stranded negative-sense RNA virus. This oligonucleotide contains an uncapped 5′ triphosphate extremity and a double-strand fragment. 3p-hpRNA sequence self-anneals to form secondary structures such hairpin or panhandle conformations. FIG. 1 shows a predicted structure 100 of 3p-hpRNA with multiple loops and double-stranded regions. Without being bound by theory, it is believed that these structural features—which distinguish viral RNA from mammalian RNA—are recognized by the RIG-I receptor. 3p-hpRNA is a specific agonist of RIG-I; it does not activate other dsRNA sensors such as TLR3 and MDA-5. 3p-hpRNA induces stronger RIG-I responses than the fully synthetic RIG-I ligand 5′ppp-dsRNA. The sequence of 3p-hpRNA is as follows: 5′-pppAGCAAAAGCAGGGUGACAAAGACAUAAUGGAUCCAA ACACUGUGUCAAGCUUUCAGGUAGAUUGCUUUCUUUGGCAUGUCCGCAAAC-3′ (87 mer) (SEQ ID NO:1).

pUUC AuK This novel RIG-I agonist is identified in this disclosure as having efficacy in slowing the growth of tumors. pUUC AuK is a single-stranded DNA sequence with an uncapped 5′ triphosphate, a double-stranded hairpin, and poly-T sequences. The sequence of pUUC AuK is as follows: 5′-pppTAATACGACTCACTATAGGCCATCCTGTTTTTTTCCCTTTTTTTTTTCT TTTTTTTTTTTTTTTTTTTTTTTTTTTTTTTTTTCTCCTTTTTTTTTCCTCTTTTTTTCCTTT TCTTTCCTTT-3′ (122 mer) (SEQ ID NO:2). As used herein, pUUC Auk includes any oligonucleotide sequence with at least 80% identity to SEQ ID NO: 2, a 5′ triphosphate or diphosphate group, at least one double-stranded region, and a poly-T or poly-U sequence of at least 13 nt.

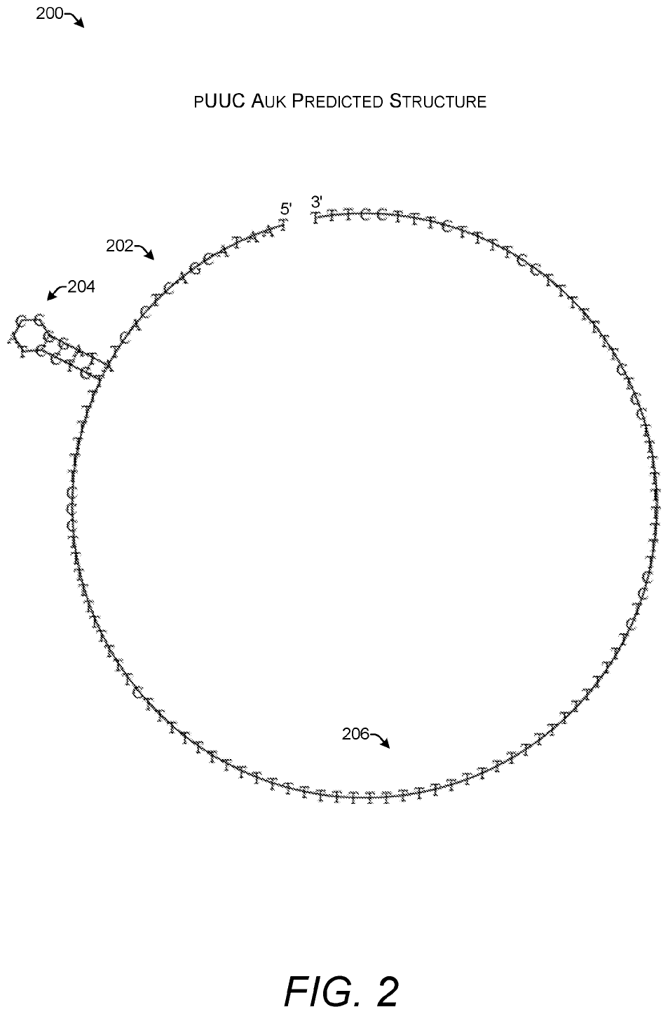

FIG. 2 shows a predicted structure 200 for pUUC AuK. The predicted structure 200 includes a first linear region 202 from the 5′ end to nucleotide 16, a hairpin region 204 from nucleotide 17 to 26, and a second linear region 206 from nucleotide 27 to the 3′ end. The first linear region 202 has the sequence as follows: 5′-pppTAATACGACTCACTAT-3′ (16 mer) (SEQ ID NO:3) The hairpin region 204 of pUUC AuK includes the nucleotides that are predicted to form a hairpin structure and has the sequence as follows: 5′-AGGCCATCCT-3′ (10 mer) (SEQ ID NO:4). The second linear region 206 has the sequence as follows: 5′-GTTTTTTTCCCTTTTTTTTTTCTTTTTTTTTTTTTTTTTTTTTTTTTTTTTTTTTTTCTCCTT TTTTTTTCCTCTTTTTTTCCTTTTCTTTCCTTT-3′ (96 mer) (SEQ ID NO:5).

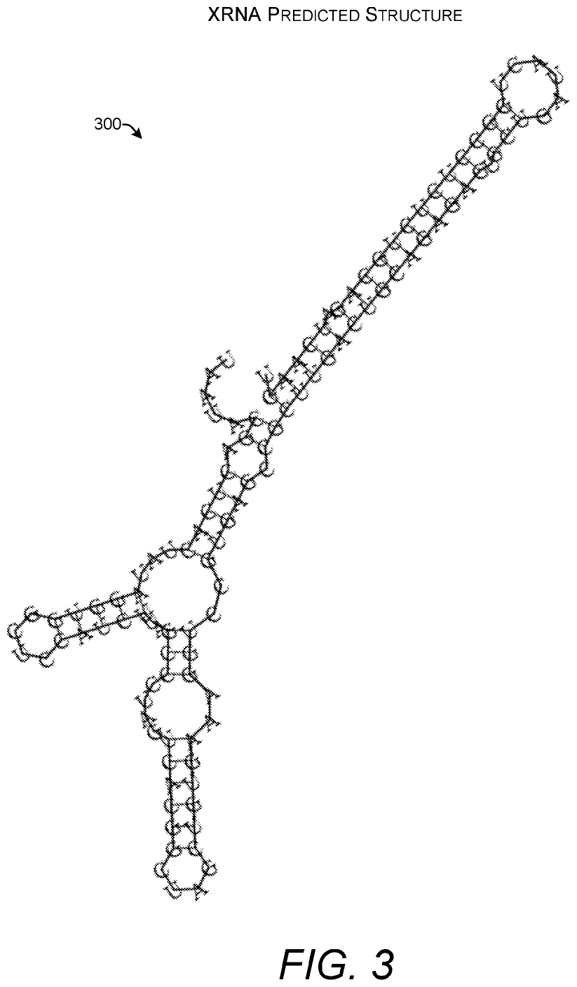

XRNA. This is an RNA sequence found in the 3′-untranslated region of the hepatitis C virus RNA genome. It is used as an RNA control because it is known to not activate RIG-I. The sequence of XRNA used in these experiments was 5′-pppUAAUACGACUCACUAUAGGUGGCUCCAUCUUAGCCCUAGUCACGGCUAGCUGU GAAAGGUCCGUGAGCCGCUUGACUGCAGAGAGUGCUGAUACUGGCCUCUCUGCAG AUCAAGU-3′ (115 mer) (SEQ ID NO:6). FIG. 3 shows a predicted structure 300 for XRNA. Although the structure has double-stranded regions, hairpins, and loops it is not an agonist for RIG-I.

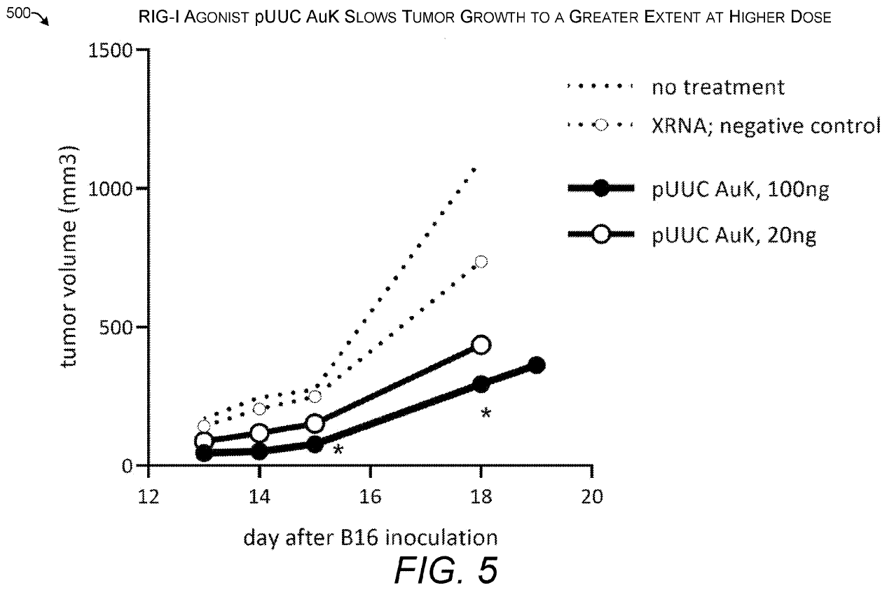

Statistical Significance. The tumor volumes shown in FIGS. 5-7 are the average (mean) volume of the tumor sizes of the mice in each treatment group (n=4 or 5). Statistically significant differences at p-value<0.05 are indicated by an asterisk symbol (*). This indicates that, for the marked time points, there is less than a 5% chance that treatment did not affect tumor growth. The statistical significance was determined by computing the standard error of the mean (SEM) between the group of mice receiving the treatment and a control group. The control group is the no treatment group for Examples 1-3 and the diluent control group for Example 4.

### Experiment 1: Treatment with 3p-hpRNA RIG-I Agonist Slows Tumor Growth

Experiment 1 shows that a RIG-I agonist can be used to slow the growth of solid tumors in a subject. The impact of treatment with a RIG-I agonist in the early stages of tumor development was studied by injecting mice with B16 cells then providing treatment one day later. Treatment with the RIG-I agonist 3p-hpRNA led to significantly slower tumor growth.

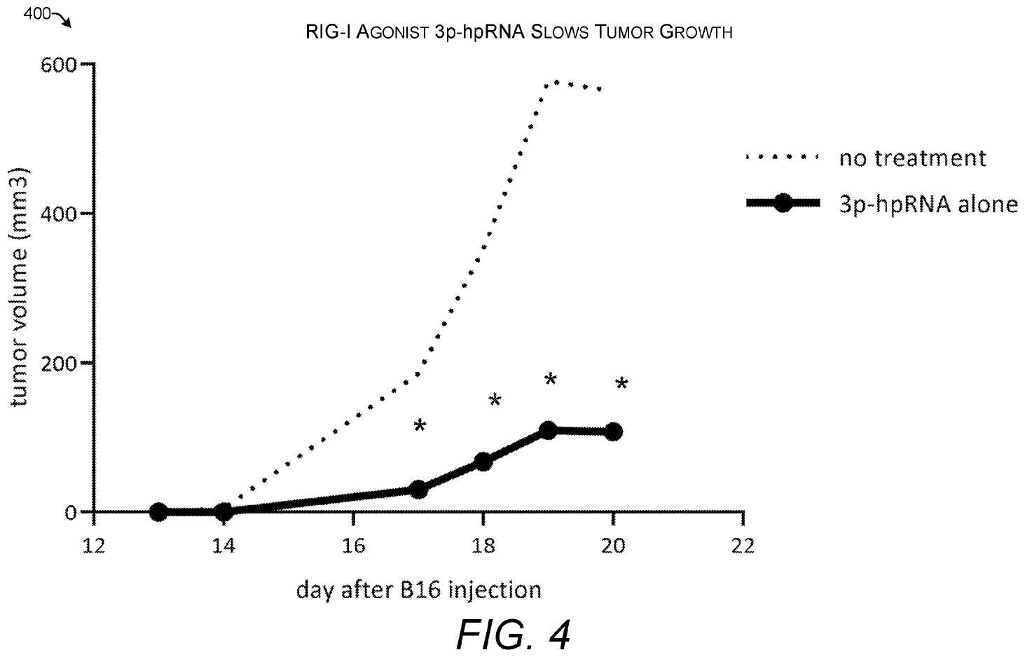

FIG. 4 is a graph 400 that shows the results of Experiment 1. The horizontal axis shows the number of days after inoculation with B16 cells. The vertical axis shows tumor volume in cubic millimeters. One day after inoculation, mice were either not treated or were injected with 100 ng of 3p-hpRNA prepared an administered as described above without an adjuvant. The tumors were measurable after 14 days. Once the tumors started growing, the tumors in the mice treated with the RIG-I agonist 3p-hpRNA (solid line) grew much slower than the tumors in untreated mice (dotted line).

### Experiment 2: pUUC AuK Exhibits Dose-Dependent Inhibition of Tumor Growth

Experiment 2 shows that treatment with pUUC AuK can also slow tumor growth. As in Experiment 1, mice were inoculated with B16 cells and treated one day later. The mice received no treatment, the negative control XRNA, a 20 ng dose of pUUC AuK or a 100 ng dose of pUUC AuK.

FIG. 5 is a graph 500 that shows the results Experiment 2. The horizontal axis shows the number of days after inoculation with B16 melanoma cells. The vertical axis shows tumor volume in cubic millimeters. Tumor growth in the mice treated with XRNA (dotted line open circles) was similar to that of the mice that received no treatment (dotted line). Treatment with pUUC AuK exhibited a dose-dependent ability to limit tumor growth. Mice treated with either 20 ng (solid line open circles) or 100 ng (solid line closed circles) of pUUC AuK showed slower tumor growth than the no-treatment group or the group treated with XRNA. The mice treated with 100 ng of pUUC AuK showed slower tumor growth than the mice treated with 20 ng of pUUC AuK.

### Experiment 3: pUUC AuK Depends on Dosage and Formulation

Experiment 3 shows that both dosage and formulation with SE change the degree to which pUUC AuK treatment slows tumor growth. As in Experiments 1 and 2, mice were inoculated with B16 cells and treated one day later. The mice received no treatment, XRNA (RNA negative control), the adjuvant SE without an agonist (adjuvant control), a 20 ng dose of pUUC AuK without an adjuvant, a 20 ng dose of pUUC AuK formulated with SE, a 4 ng dose of pUUC AuK without an adjuvant, or a 4 ng dose of pUUC AuK formulated with SE.

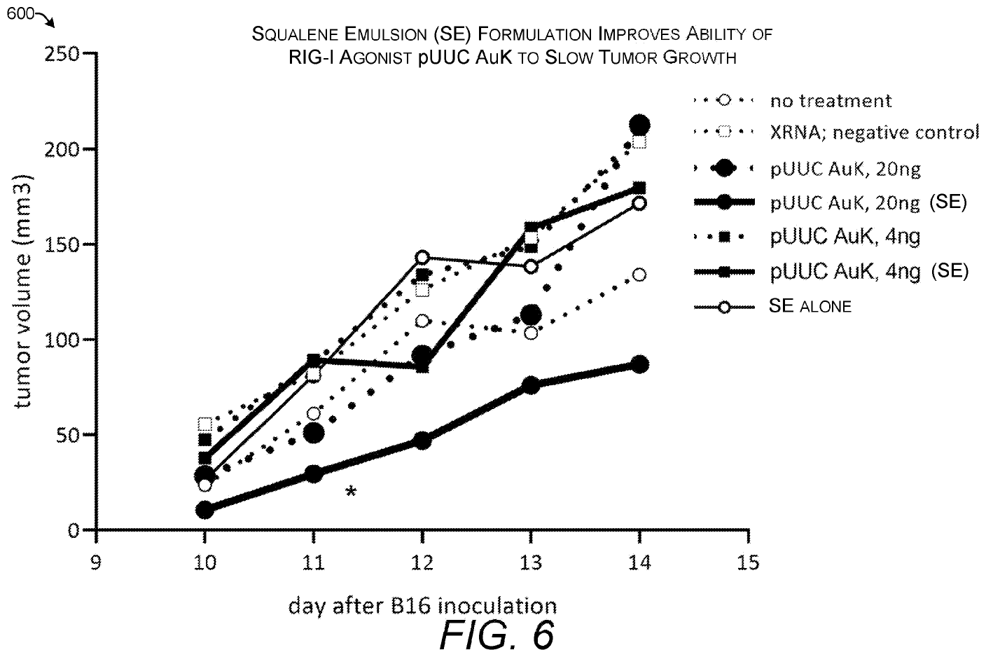

FIG. 6 is a graph 600 that shows the results of Experiment 3. The horizontal axis shows the number of days after inoculation with B16 cells. The vertical axis shows tumor volume in cubic millimeters. Tumor growth proceeded approximately at the same rate for all treatments other than 20 ng of pUUC AuK with a SE adjuvant (solid line closed circles). This indicates that formulation with a SE adjuvant can result in slower tumor growth for dosages pUUC AuK that otherwise would not have a strong effect on the rate of tumor growth.

### Experiment 4: Treatment of Established Tumors with pUUC AuK Slows Growth

Experiment 4 shows that treatment of established tumors measuring approximately 200 mm³ with the RIG-I agonist pUUC AuK slows subsequent tumor growth. Mice were inoculated with B16 cells then seventeen days later were either injected with 5% dextrose water (diluent control), SE without an agonist (adjuvant control), or with 100 ng pUUC AuK formulated with SE as described above.

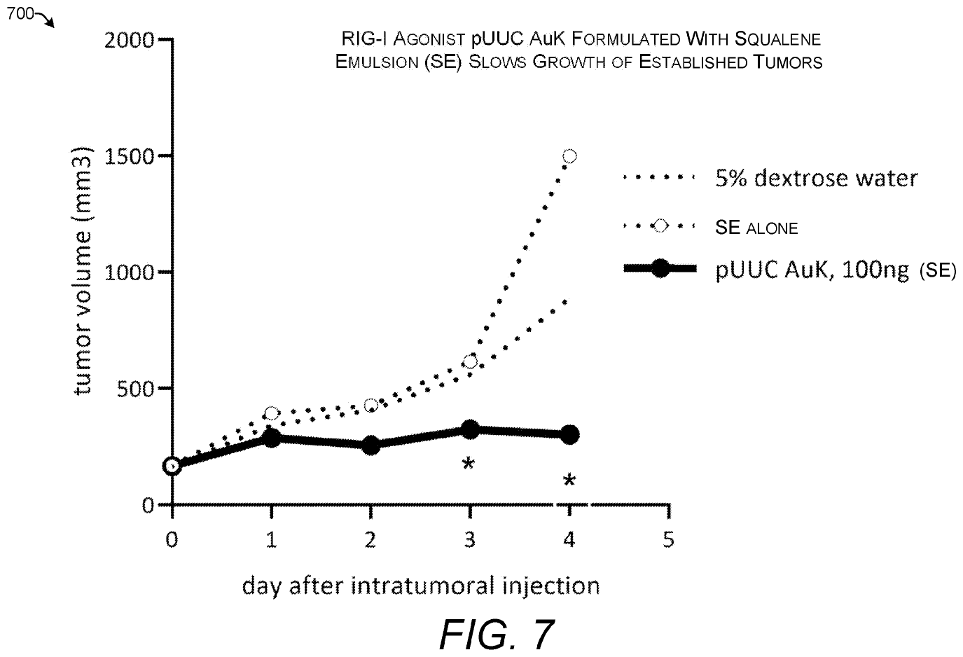

FIG. 7 is a graph 700 that shows the results of Experiment 4. The horizontal axis shows the number of days after intratumoral injection of either a control or a treatment. Day 0 on the horizontal axis represents 17 days after inoculation with B16 cells. The vertical axis shows tumor volume in cubic millimeters. Treatment with a formulation of 100 ng of pUUC AuK and SE (solid line closed circles) limited further tumor growth while the 5% dextrose water (dotted line) and SE alone (dotted line open circles) did not.

### CONCLUSION

Although the subject matter has been described in language specific to structural features and/or methodological acts, it is to be understood that the subject matter defined in the appended claims is not necessarily limited to the specific features or acts described above. Rather, the specific features and acts are disclosed as example forms of implementing the claims.

The terms “a,” “an,” “the,” and similar referents used in the context of describing the invention are to be construed to cover both the singular and the plural unless otherwise indicated herein or clearly contradicted by context. The terms “based on,” “based upon,” and similar referents are to be construed as meaning “based at least in part” which includes being “based in part” and “based in whole,” unless otherwise indicated or clearly contradicted by context. The terms “portion,” “part,” or similar referents are to be construed as meaning at least a portion or part of the whole including up to the entire noun referenced. As used herein, “approximately” or “about” or similar referents denote a range of ±10% of the stated value.

The various implementations described herein are not limiting nor is every feature from any given implementation required to be present in another implementation. Any two or more of the implementations may be combined together unless context clearly indicates otherwise. As used herein in this document “or” means and/or. For example, “A or B” means A without B, B without A, or A and B. As used herein, “comprising” means including all listed features and potentially including addition of other features that are not listed. “Consisting essentially of” means including the listed features and those additional features that do not materially affect the basic and novel characteristics of the listed features. “Consisting of” means only the listed features to the exclusion of any feature not listed.

Certain embodiments are described herein, including the best mode known to the inventors for carrying out the invention. Of course, variations on these described embodiments will become apparent to those of ordinary skill in the art upon reading the foregoing description. Skilled artisans will know how to employ such variations as appropriate, and the embodiments disclosed herein may be practiced otherwise than specifically described. Accordingly, all modifications and equivalents of the subject matter recited in the claims appended hereto are included within the scope of this disclosure. Moreover, any combination of the above-described elements in all possible variations thereof is encompassed by the invention unless otherwise indicated herein or otherwise clearly contradicted by context.

Furthermore, references have been made to publications, patents and/or patent applications throughout this specification. Each of the cited references is individually incorporated herein by reference for its particular cited teachings as well as for all that it discloses.

1. A formulation for slowing growth of tumors in a subject comprising an effective amount of a RIG-I agonist comprising pUUC AuK and an adjuvant.

2. The formulation of claim 1, wherein the adjuvant is a nanostructured lipid carrier (NLC), a squalene emulsion (SE), a GLA-AF adjuvant (aqueous nanosuspension of GLA), or an aluminum adjuvant (alum).

3. The formulation of claim 2, wherein the adjuvant is the SE and the SE comprises squalene or synthetic squalene, dextrose, polysorbate 80, and lecithin.

4. A formulation for slowing growth of tumors in a subject comprising an effective amount of a RIG-I agonist comprising an oligonucleotide with at least one double-stranded region and at least one 5′ phosphate combined with a squalene emulsion (SE) adjuvant.

5. The formulation of claim 4, wherein the adjuvant is the SE and the SE comprises squalene or synthetic squalene, dextrose, polysorbate 80, and lecithin.

6. The formulation of claim 4, wherein the RIG-I agonist comprises 3p-hpRNA or pUUC AuK.

7. The formulation of claim 5, wherein the RIG-I agonist comprises a sequence having at least 90% identity with SEQ ID NO:1.

8. The formulation of claim 1, wherein the RIG-I agonist comprises a sequence having at least 90% identity with SEQ ID NO:2.

9. The formulation of claim 1, wherein the RIG-I agonist comprises a hairpin region sequence having SEQ ID NO:4.

10. The formulation of claim 1, wherein the RIG-I agonist comprises a sequence having at least 90% identity with a hairpin region having SEQ ID NO:4 and at least 80% identity with a linear region having SEQ ID NO:3.

11. The formulation of claim 1, wherein the RIG-I agonist comprises a first linear region having a length of 14-18 nucleotides, a hairpin region having a length of 8-12 nucleotides, and a second linear region having a length of 90-100 nucleotides.

12. The formulation of claim 11, wherein the first linear region comprises a sequence having at least 80% identity with SEQ ID NO:3.

13. The formulation of claim 11, wherein the hairpin region comprises a sequence having at least 90% identity with SEQ ID NO:4.

14. The formulation of claim 11, wherein the second linear region comprises a sequence having at least 80% identity with SEQ ID NO:5.

15. The formulation of any of claim 1, wherein the effective amount of the RIG-I agonist is between about 4 ng and about 120 ng, between about 20 ng and about 120 ng, between about 90 ng and 110 ng, or about 100 ng.

16. (canceled)

17. (canceled)

18. (canceled)

19. The formulation of claim 1, wherein an effective amount of the RIG-I agonist is an amount that induces an immune response in the subject.

20. The formulation of claim 19, wherein an effective amount of the RIG-I agonist is an amount that activates a RIG-I receptor in the subject.

21. The formulation of claim 20, wherein an effective amount of the RIG-I agonist is an amount that triggers signaling cascades that lead to the production of type I IFNs and pro-inflammatory cytokines in the subject.

22. The formulation of claim 1, further comprising one or more excipients.

23. A method for slowing growth of solid tumors in a subject comprising administering an effective amount of the formulation of any of claim 1 to the subject.

24. (canceled)
